(19) United States
(12) Patent Application Publication
(10) Pub. No.: US 2022/0208360 A1
(43) Pub. Date: Jun. 30, 2022

(54) ELECTRONIC COORDINATION OF HEALTHCARE AND ASSOCIATED DISEASE REGISTRY

(71) Applicant: Nova Southeastern University, Fort Lauderdale, FL (US)

(72) Inventors: Julie A. Jacko, Fort Lauderdale, FL (US); Johannes Vieweg, Fort Lauderdale, FL (US); François Sainfort, Fort Lauderdale, FL (US); Farzanna Haffizulla, Fort Lauderdale, FL (US)

(73) Assignee: Nova Southeastern University, Fort Lauderdale, FL (US)

(21) Appl. No.: 17/601,107

(22) PCT Filed: Apr. 2, 2020
(86) PCT No.: PCT/US20/26399

§ 371 Date: Oct. 4, 2021

Related U.S. Application Data
(60) Provisional application No. 62/828,089, filed on Apr. 2, 2019.

(51) Int. Cl. G16H 40/20; G16H 10/60

(52) U.S. Cl. CPC G16H 40/20; G16H 10/60

(57) ABSTRACT

The invention provides systems and methods for electronic coordination of healthcare. This healthcare coordination system is a digital platform for health providers and patients, providing user-friendly tools to navigate the continuum of healthcare services, particularly in chronic disease, such as diabetes or cancer. The invention also provides databases specific to a particular disease and/or population.

### FIELD OF THE INVENTION

The invention is encompassed within the fields of computer science, medicine, and healthcare and generally relates to patient navigation of the healthcare system, particularly to the use of electronic systems for management of healthcare, and mostly particularly to systems and methods for electronic coordination of healthcare between patients, patient navigators, and healthcare providers.

### BACKGROUND

The healthcare system in the United States is extensive and complicated. Patient navigators, both human and/or electronic, work to mitigate the complications. A patient navigator helps to guide a patient through the healthcare system (as defined by the National Cancer Institute). This includes help going through screening, diagnosis, treatment, and follow-up of a medical condition, such as cancer or diabetes. A patient navigator helps patients to communicate with their healthcare providers, so they get the information they need to make decisions about their healthcare. Patient navigators may also help patients set up appointments for doctor visits and medical tests and to get financial, legal, and social support. They may also work with insurance companies, employers, case managers, lawyers, and others who have an effect on a patient's healthcare needs.

A recent systemic review of patient navigators for people with chronic diseases can be found in databases of the National Institute of Health. The instant inventors conducted this systemic review. The review included original reports of randomized controlled trials of patient navigator programs compared to usual care of adult and pediatric patients with any one of a defined set of chronic diseases. From a total of 14,672 abstracts, 67 unique studies fit the inclusion criteria. Of these, 44 were in cancer, 8 in diabetes, 7 in HIV/AIDS, 4 in cardiovascular disease, 2 in chronic kidney disease, 1 in dementia, and 1 in patients having more than one condition. Findings indicate that patient navigator programs improve processes of care. However, few studies assessed patient experience, clinical outcomes, or costs. The patient navigation in diabetes was very limited in this regard. The inability to definitely outline successful components remains a key uncertainty in the use of patient navigator programs across chronic diseases.

The instant inventors formed a clinician-led task force of physicians and community experts in South Florida focused on diabetes to uncover unmet needs in the care continuum, identify areas of improvement for coordinating care across the continuum, and patient ability to effectively access specialty care. The task force examined implementing new ways of empowering, supporting, and involving patients while placing them at the center of care along the continuum. The task force additionally explored opportunities for digital health to support clinicians, patients, and caregivers. The ultimate goal of the task force was to catalyze innovation and generate solutions for high quality and affordable care. There was strong consensus among the clinicians that developing a novel “health GPS” to support patients and providers in navigating the full continuum of the healthcare system would be innovative and impactful.

### SUMMARY OF THE INVENTION

The instant invention provides such a “health GPS.” This inventive healthcare coordination system is a digital platform for health providers and patients, providing user-friendly tools to navigate the continuum of healthcare services, particularly in, but not limited to, chronic disease, such as diabetes or cancer. It is a complete solution for what is currently a system of fragmented care, inefficient processes and systems, disenfranchised patients, and frustrated clinicians. It is designed to be a guiding force for the seamless and timely flow and movement of patients, caregivers, data, and communication through a complex system of care. These tools allow for: direct messaging between patients and caregivers; a community directory for social services; and patient portals (enabling patients better access to their own information). The tools, packaged into a complete coordinated system (illustrated schematically in FIG. 1), facilitate access to specialty care and coordinates primary and specialty care to fully and effectively integrate and guide navigation across the continuum of care.

The terms “healthcare coordinating system”, “healthcare coordination system” “health GPS”, “patient navigator”, and “patient navigator system” are used interchangeably herein.

In a most basic aspect, the invention provides a new modality for managing healthcare using electronic organization.

In one aspect, the invention provides a system for electronic coordination of healthcare having a digital platform accessible for patients and healthcare providers through the internet. Healthcare providers can include medical personnel and any other people having an effect on the healthcare needs of a patient, such as, but not limited to employers, lawyers, insurance companies, and case managers. The digital platform of the inventive system includes a plurality of tools for navigating a continuum of healthcare services. The digital platform is supported by architecture comprising a plurality of tiers, such as, but not limited to a web tier, a business tier, a data tier, an analytics tier, a security management tier, and an application management tier. The data tier can include access to external data sources including, but not limited to, electronic medical records (EMR), insurance provider databases, and healthcare payer systems.

The inventive system is particularly useful to patients having chronic diseases, such as, but not limited to cancer and diabetes. Additionally, the inventive system can be used to create a “disease registry” specific for a chronic disease and/or a specific population or group of people.

Other objectives and advantages of this invention will become apparent from the following description, wherein are set forth, by way of example, certain embodiments of this invention.

### BRIEF DESCRIPTION OF THE DRAWINGS

A more complete understanding of the present invention may be obtained by references to the accompanying drawings when considered in conjunction with the subsequent detailed description. The embodiments illustrated in the drawings are intended only to exemplify the invention and should not be construed as limiting the invention to the illustrated embodiments.

FIG. 1 is a schematic depiction of the coordinating structure and use of the inventive healthcare coordination system.

FIG. 2 is a schematic depiction of the architecture needed to support the inventive healthcare coordination system.

FIG. 3 is a schematic depiction of the architecture needed to support the Caribbean Cancer Data Registry.

FIG. 4 is a schematic depiction of the registry builder of the Caribbean Cancer Data Registry.

FIG. 5 is a schematic depiction of data-in-transit through the inventive healthcare coordination system.

FIG. 6 is a schematic depiction of data types at rest in the inventive healthcare coordination system.

FIG. 7 is a schematic depiction of the data registry security for the inventive healthcare coordination system.

FIG. 8 is a prior art figure illustrating a theoretical model of interconnecting mechanisms underlying associations between socioeconomic status (SES) and health. See Norton et al. J Am Soc Nephrol 27(9):2576-2595 2016.

### DETAILED DESCRIPTION OF THE INVENTION

For the purpose of promoting an understanding of the principles of the invention, reference will now be made to embodiments illustrated herein and specific language will be used to describe the same. It will nevertheless be understood that no limitation of the scope of the invention is thereby intended. Any alterations and further modification in the described systems and methods for electronic coordination of healthcare along with any further application of the principles of the invention as described herein, are contemplated as would normally occur to one skilled in the art to which the invention relates.

### System Architecture

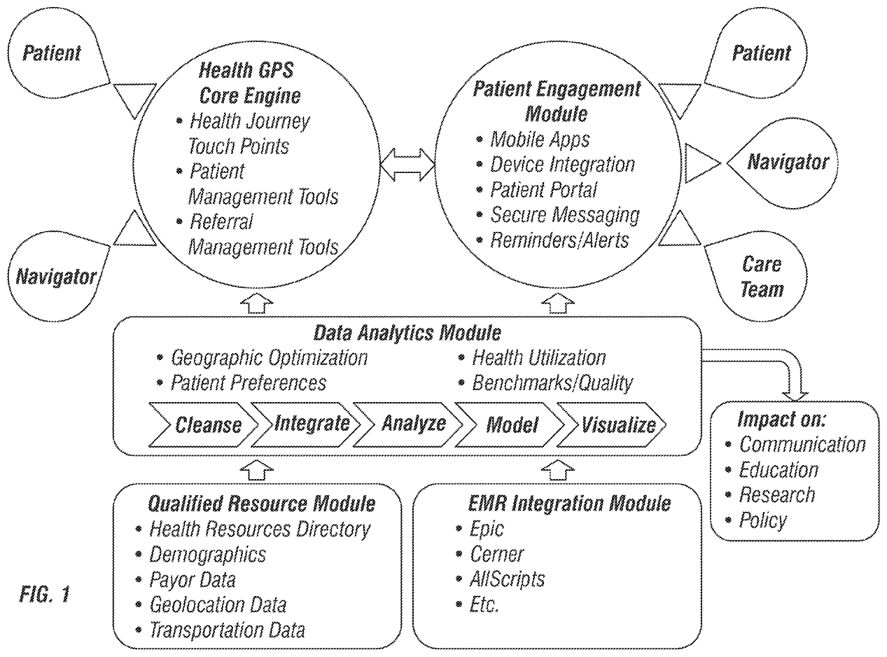

The inventive healthcare coordination system “health GPS” is a digital platform for health providers and patients, providing user-friendly tools to navigate the continuum of healthcare services, particularly in chronic disease, such as diabetes or cancer. Packaged into a complete coordinated system (illustrated schematically in FIG. 1), the inventive “health GPS” facilitates access to specialty care and coordinates primary and specialty care to fully and effectively integrate and guide navigation across the continuum of care.

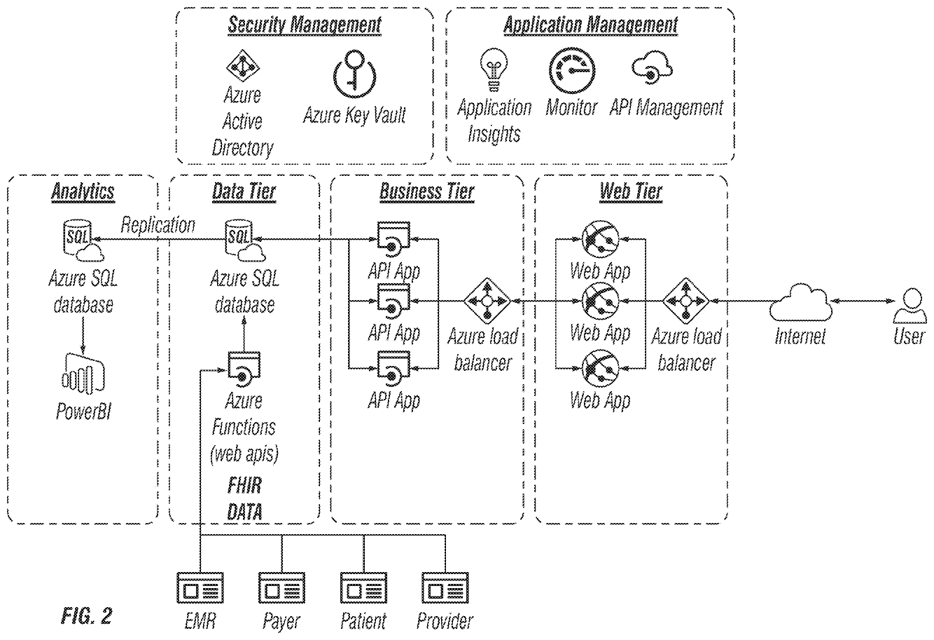

The architecture needed to support the Patient Navigator is comprised of six tiers managed by NSU's (Applicant) Office of Innovation and Information Technology and supported by Microsoft's Azure Database and Security platform. The architectural tiers are comprised of the following: Web Tier, Business Tier, Data Tier, and Analytics. Security and Application Management would occur throughout the tiers. As shown in FIG. 2, a user would access the application through the internet and the request would pass through the tiers for data entry or retrieval.

### Web Tier

The web tier contains the web application, supported by Internet Information Services (IIS) for Windows. The website would be built using Html5 and ReactJs which is a JavaScript framework. In order to retrieve and send data, calls to the Web Application Programming Interface (API) will be completed.

Three Web Applications are needed to provide redundancy to support a quality user experience through utilization of the Azure load balancer. The load balancer will balance the distribution of each request between the web applications, so that that user experience is not inhibited. As the user accesses the Patient Navigation system through the web application, the load balancer will manage the request accordingly to reduce latency.

### Business Tier

The Business tier will consist of three Web API apps in place for redundancy along with an Azure load balancer to manage each request and reduce latency for the user experience. The Web API provides the interface into the business logic, which will validate the user request against the business logic requirement. Once the data is validated within the Business tier it is sent to the database management systems, handled within the Data Tier.

### Data Tier

The Data tier contains the Azure SQL database management system, which is where the data for the system will be stored securely and in compliance with all federal and state guidelines. Information into the system will be updated from multiple data sources. Data from external sources will be updated through Azure Functions, which is a service provided by Azure that allows the ability to build serverless web APIs that are secure and scalable, based on demand. External data sources will include information from systems such as Electronic Medical Records (EMR), Insurance Providers or Healthcare Payer systems, Patient Databases, and Providers. This data will conform with Fast Healthcare Interoperability Resources (FHIR) standards. For external data sources that does not conform with FHIR standards, the system according to the invention collects and converts the external data sources to conform with FHIR standards.

### Analytics

Analytics tier will contain another Azure SQL database. The information stored into this database system will come directly from the data tier and will be utilized for analytical reporting and dashboards. For dashboards and reports, the system will be utilizing Microsoft PowerBI.

### Security Management

Security Management will be handled through Azure Active Directory (Azure AD) to manage authentication into the system, along with the Azure Key Vault.

Azure AD manages user identities and creates intelligence-driven access policies to control access to data sources, based on specific roles such as administrator, contributor, and user. Utilizing Azure AD, Multi-Factor Authentication (MFA) will be added as a secondary level of authentication.

To secure the transfer of data calls within the API app, the Azure Key Vault will be utilized to store private information such as passwords, which are encrypted and can be audited.

### Application Management

Application Management will be operated utilizing Application Insights, Azure Monitor, and API Management applications. Application Insights is used to monitor the performance of the application and provide the ability to trace or identify issues as they arise. Application Insights is an extensible Application Performance Management (APM) service for web developers and used to monitor live web applications. It will automatically detect performance anomalies. It includes analytics tools to help diagnose issues and to understand what users are doing on the application. It supports continuous performance improvement and usability.

Azure Monitor integrates the capabilities of Log Analytics and Application Insights for end-to-end monitoring of the web application and its system. It monitors the application, infrastructure and network.

API Management allows APIs to be published, managed, secured and analyzed. It provides the ability to manage and secure the APIs. It provides three methods of securing data passing through the API such as keys, tokens, and IP filtering. API Management will also allow for auditing the type of API calls being made.

### Infrastructure Security

Access to the application will be authenticated using Azure AD. Views to the application will be based on the user's permissions/roles managed through the OAuth 2.0 framework. OAuth is an open standard for authorization. OAuth provides applications a ‘secure delegated access’ to server resources on behalf of a resource owner. It specifies a process for resource owners to authorize third-party access to server resources without sharing their credentials.

The Web and API application will be utilizing Secure Sockets Layer (SSL) for transmission of data. SSL is a standard security technology for establishing an encrypted link between a server and a client, such as a (website) and a browser, or a mail server and a mail client (e.g., Outlook). SSL allows sensitive information such as credit card numbers, social security numbers, and login credentials to be transmitted securely.

All data stored within the Azure SQL database will be encrypted using an inherit feature within the Azure database. The feature makes encryption transparent to applications. It also allows encryption of sensitive data inside client applications without sharing the encryption keys with SQL Database. Azure SQL databases can be tightly secured to satisfy most regulatory or security requirements, including HIPAA, ISO 27001/27002, and PCI DSS Level 1.

### Summary of System Architecture

Many details of the web application architecture are supported by existing and proven platforms within the Microsoft framework. It is important to understand that all of the technology components and interfaces use mainstream standards widely adopted across the globe by many healthcare solution providers. This sound architecture will provide key attributes such as: flexibility, scalability, elasticity and security. These attributes will allow for the platform to grow overtime. Therefore, as the constituent and use-cases evolve over time, so will this solution.

### Disease Registry

The inventive healthcare coordination system can be used to create databases specific to a particular disease and/or population. One example of such a database includes cancer among people of Caribbean descent.

### Caribbean Cancer Data Registry Project

The purpose of this project is to develop and implement a regional Caribbean cancer registry resource as a catalyst for future development of targeted interventions for disease prevention, cancer screenings, and health education that aligns with Caribbean culture and ethnic preferences. Specifically, the system infrastructure will be developed and implemented for the data repository with the following aims:

1. Design, develop, and implement the first community-based registry and resource repository infrastructure to host epidemiologic and social determinant data of the most prevalent malignancies in Caribbean immigrants.

2. Design secure electronic tools to support data collection.

3. Design a secure, role-based dashboard system to provide data and analytics access to researchers.

### Data and Systems Governance

The data captured in the registry will be owned by the Dr. Kiran C. Patel College of Allopathic Medicine's Department of Population Health and will constitute a qualified data resource for clinicians and researchers who have sought and received data privileges for access to the HIT infrastructure that hosts the Caribbean Registry. Researchers who publish results that were enabled through the use of the registry will be required to acknowledge the funding source that enabled the construction and data population of the registry. The overall vision of, and specifications for the Caribbean Registry and related HIT infrastructure are led by the Chair of the Department of Population Health for NSU MD, Dr. Julie Jacko, one of the instant inventors, who is Director of the System & Infrastructure Core on this project. She will have oversight of the System Development Team, which will be housed in NSU's Office of Innovation and Information Technology (OIIT). This reflects the collaborative nature of this endeavor, which combines the collective strengths of the Department of Population Health, with those of OIIT. The initial data elements chosen to populate the registry have been derived in consultation with the PI, Dr. Haffizulla, and with the Director of the Data Collection & Analytics Core, Dr. Sainfort. The anticipated expansion of the set of data elements captured in the registry will be conducted by these project leaders, in consultation with the Scientific Advisory Board.

The following paragraphs provide the technical detail of the registry builder and overall infrastructure to be built in this project, including a conceptual depiction of the registry builder, the architectural overview, security management, application management, and infrastructure security. The project team recognizes that security is of utmost importance and hence, every detail of the architecture has been designed and will be implemented with security as the top priority.

### The Registry Builder

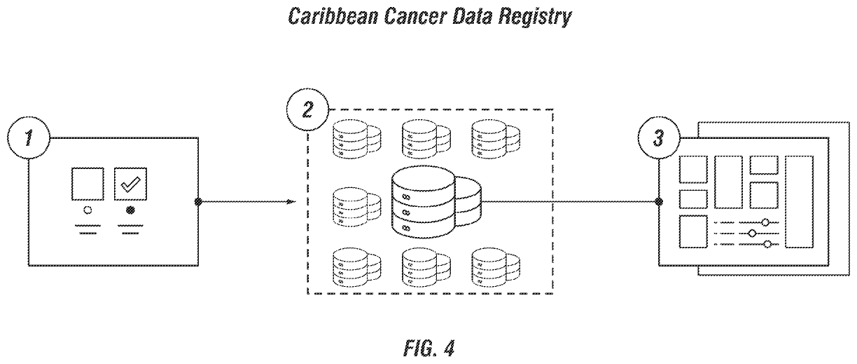

FIG. 4 shows a conceptual depiction of the registry builder. This registry builder allows researchers the ability to define a registry template or select from a pre-made template to define data collection. The template for this project will be defined to reflect the initial set of data elements identified for the project, and it will be refined and expanded as described, over time. The project personnel performing data entry (i.e., the PI, abstractors and project coordinator) will input individual patient records and surveys using the form generated by the template system. It will also be possible to upload a .csv file to batch process a large amount of records (step 1, FIG. 4).

While it is being populated and once it is complete, the new data registry will be stored as a new, secure, qualified data resource in a data lake that can now be activated for access as part of an integrations hub. The integrations hub acts as an App Store and allows easy sharing of data sets with the community of researchers using the resource (step 2, FIG. 4). As also shown in step 2, the data set can be private and unverified when first created or during creation. This provides the ability to leverage the data independently. The sharing of data sets allows verification process that will validate the data sets. Once the data registry is available in the secure data lake, it can be mapped to multiple dashboards for analytics (step 3, FIG. 4).

### Architecture Overview

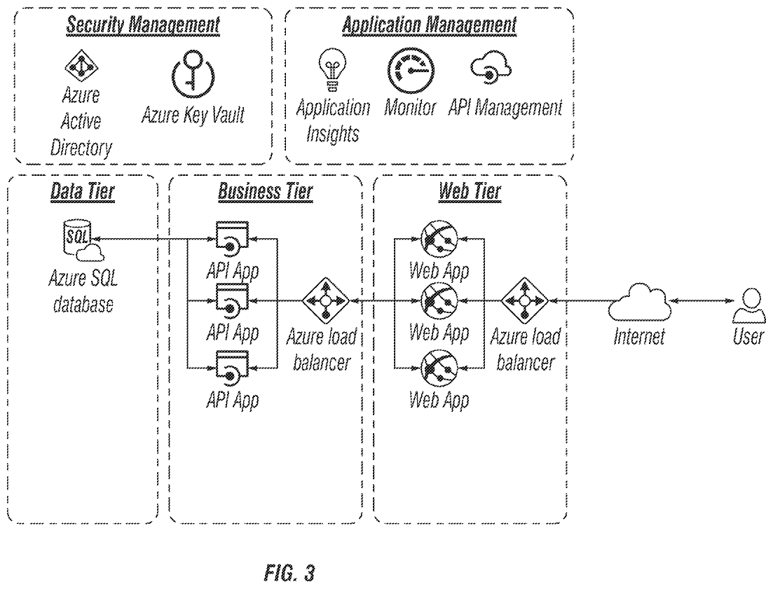

The architecture needed to support the Caribbean Registry is comprised of six tiers managed by the System Development Team and supported by Microsoft's Azure Database and Security platform. The architectural tiers are comprised of the following tiers: Web Tier, Business Tier, and Data Tier. Security and Application Management will occur throughout the tiers. FIG. 3 depicts the Caribbean Cancer Data Registry's overall architecture. As shown, a user will access the application through the Internet and the request will pass through the three tiers for data entry or retrieval.

The web tier contains the web application, supported by Internet Information Services (IIS) for Windows. The website will be built using Html5 and ReactJs which is a JavaScript framework. In order to retrieve and send data, calls to the Web Application Programming Interface (API) will be completed. Three Web Applications are needed to provide redundancy to support a quality user experience through utilization of the Azure load balancer. The load balancer will balance the distribution of each request between the web applications, so that that user experience is not inhibited. As the user accesses the Caribbean Registry system through the web application, the load balancer will manage the request accordingly to reduce latency.

The Business tier will consist of three Web API apps in place for redundancy along with an Azure load balancer to manage each request and reduce latency for the user experience. The Web API provides the interface into the business logic, which will validate the user request against the business logic requirement. Once the data is validated within the Business tier it is sent to the database management systems, handled within the Data Tier.

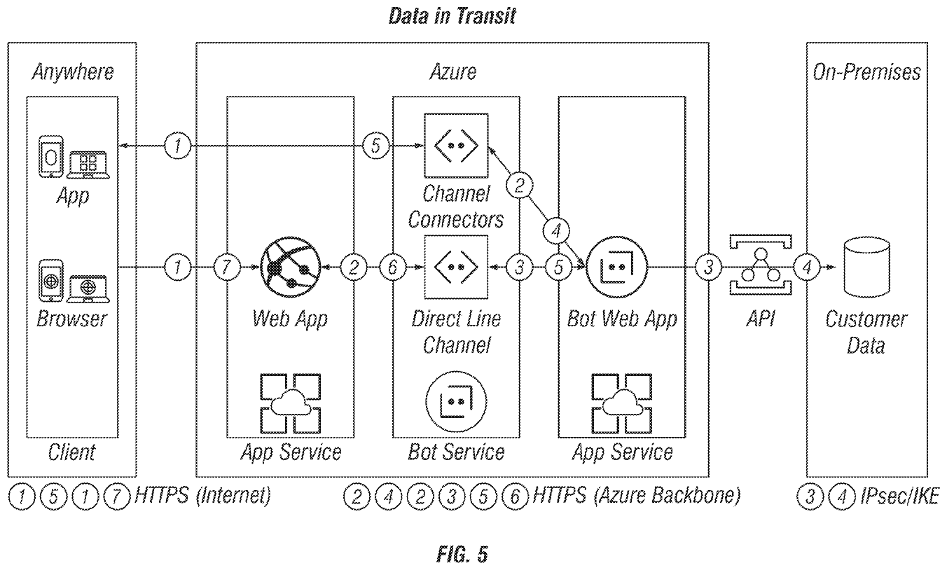

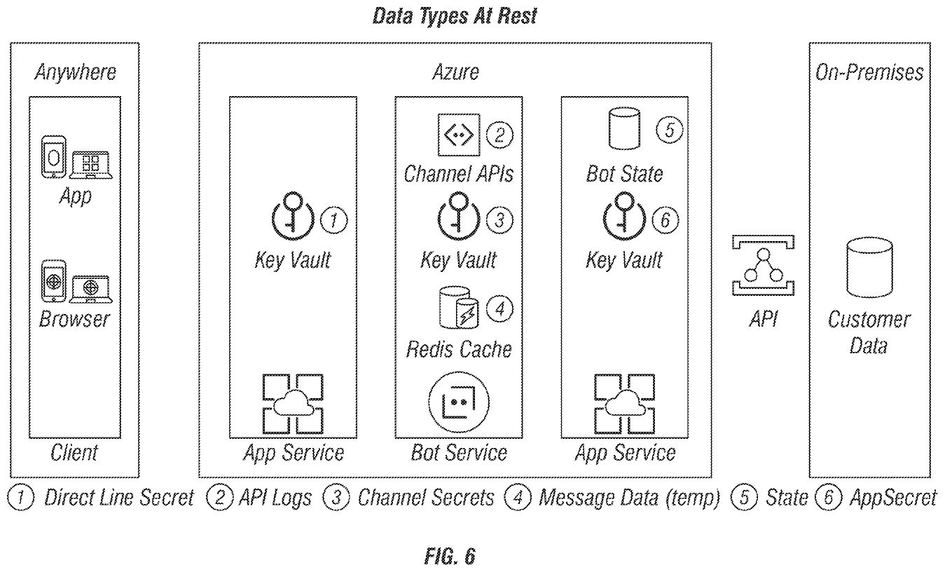

The Data tier contains the Azure SQL database management system, which is where all the Caribbean registry data entered by the PI, the abstractors, or project coordinator will be stored. The database management system provides the ability to encrypt the data stored within the tables. FIGS. 5 and 6 show the data-in-transit and the data types at rest, respectively.

### Security Management

Security Management will be handled through Azure Active Directory (Azure AD) to manage authentication into the Caribbean Registry, along with the Azure Key Vault.

Azure AD manages user identities and creates intelligence-driven access policies to control access to data sources, based on specific roles such as administrator, contributor, and user. Utilizing Azure AD, Multi-Factor Authentication (MFA) will be added as a secondary level of authentication. In order to secure the transfer of data calls within the API app, the Azure Key Vault will be utilized to store private information such as passwords, which are encrypted and can be audited.

### Application Management

Application Management will be operated utilizing Application Insights, Azure Monitor, and API Management applications. Application Insights is used to monitor the performance of the application and provide the ability to trace or identify issues as they arise. Application Insights is an extensible Application Performance Management (APM) service for web developers and used to monitor live web applications. It will automatically detect performance anomalies. It includes analytics tools to help diagnose issues and to understand what users are doing on the application. It supports continuous performance improvement and usability. Azure Monitor integrates the capabilities of Log Analytics and Application Insights for end-to-end monitoring of the web application and its system. It monitors the application, infrastructure and network. API Management allows APIs to be published, managed, secured and analyzed. It provides the ability to manage and secure the APIs. It provides three methods of securing data passing through the API such as keys, tokens, and IP filtering. API Management will also allow for auditing the type of API calls being made.

### Infrastructure Security

Access to the application will be authenticated using Azure AD. Views to the application will be based on the users' permissions/roles managed through the OAuth 2.0 framework. OAuth is an open standard for authorization. OAuth provides applications a ‘secure delegated access’ to server resources on behalf of a resource owner. It specifies a process for resource owners to authorize third-party access to server resources without sharing their credentials. The Web and API application will be utilizing Secure Sockets Layer (SSL) for transmission of data. SSL is a standard security technology for establishing an encrypted link between a server and a client, such as a (website) and a browser, or a mail server and a mail client (e.g., Outlook). SSL allows sensitive information such as credit card numbers, social security numbers, and login credentials to be transmitted securely.

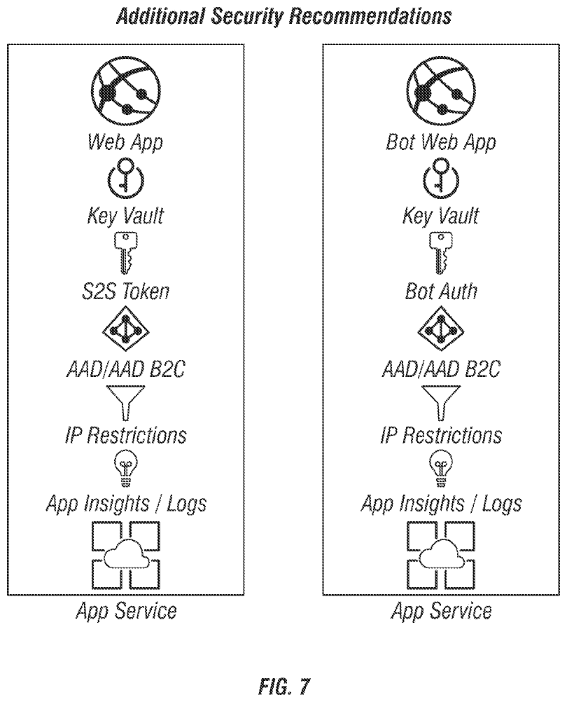

All data stored within the Azure SQL database will be encrypted using an inherent feature within the Azure database. The feature makes encryption transparent to applications. It also allows encryption of sensitive data inside client applications without sharing the encryption keys with SQL Database. Azure SQL databases can be tightly secured to satisfy most regulatory or security requirements, including HIPAA, ISO 27001/27002, and PCI DSS Level 1. FIG. 7 depicts the security recommendations for the system to ensure that it is functioning according to the highest standards possible.

Security Recommendation (FIG. 7, left)—1. store direct line secret in key vault; 2. generate token server-side to protect direct line secret client-side; 3. require auth to Web App for sensitive use cases; 4. restrict IP access to Web App for internal-facing use cases; 5. log user access and detect anomalies; and 6. use regional Direct Line domain.

Security Recommendation (FIG. 7, right)—1. store App secret in key vault; 2. enable bot authentication and disable bot emulator flag; 3. Send AAD auth card for sensitive commands/queries; 4. restrict IP access to bot Web App to regional channel; and 5. log utterances for anomaly detection and investigation.

### Cancer Registry

Many details of the web application architecture are supported by existing and proven platforms within the Microsoft framework. It is important to understand that all of the technology components and interfaces use mainstream standards widely adopted across the globe by many healthcare solution providers. This sound architecture will provide key attributes such as: flexibility, scalability, elasticity and security. These attributes will allow for the platform to grow overtime. Therefore, as the constituent and use-cases evolve over time, so will this solution.

### Utilization of the Caribbean Cancer Data Registry

After the system infrastructure is built, data collection tools will be implemented and tested. The data repository will be progressively populated with valid and reliable data from the Caribbean immigrant population. This data will be extracted and leveraged from multiple clinical sites.

As these multiple clinical sites may each have their own format for storing the data, another aspect of the invention includes a system that collects, converts and consolidates patient information from various physicians and health-care providers into a standardized format, stores the information in network-based storage devices, and optionally provides notifying messages to health care providers and/or patients whenever that information is updated.

Whenever the patient information is updated, it will first be converted into the standardized format and then stored in the collection of medical records on one or more of the network-based storage devices. In this regard, the patient information can be updated and used in the registry template set up as shown in FIG. 4 and discussed in the related text. After the updated information about the patient's condition has been stored in the collection, the content server, which is connected to the network-based storage devices, generates a message containing the updated information. This message is transmitted in a standardized format over the computer network to all physicians and health-care providers that have access to the patient's information so that all users can quickly be notified of any changes without having to manually look up or consolidate all of the providers' updates.

An exploratory data analysis examining relationships between clinical data, social determinant factors, and health outcomes will be performed.

As stated by Penman-Aguilar et al. (J Public Health Manag Pract Suppl 1:S33-42 2016) in the Abstract “Reduction of health disparities and advancement of health equity in the United States (are of the utmost importance) require high quality data indicative of where the nation stands vis-à-vis health equity, as well as proper analytic tools to facilitate accurate interpretation of this data.” Text in parentheses added. Although much is understood about the role of social determinants of health in shaping the health of populations, little to no data have been collected regarding the fast-growing Caribbean population in Florida and the rest of the United States. Researchers need the data collected over time, via the project described herein, to advance understanding of the pathways through which they operate on particular health outcomes.

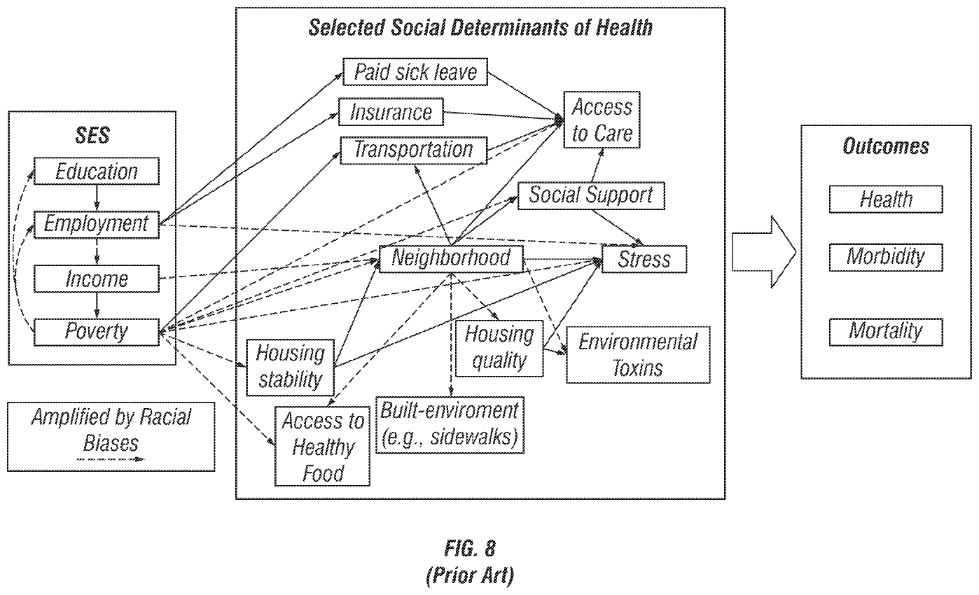

In their extensive systematic review of health disparities, Norton et al. (J Am Soc Nephrol 27(9):2576-2595 2016) developed a theoretical model (FIG. 8) depicting the interconnected mechanisms underlying associations between socioeconomic status/factors (SES) and health. They further demonstrate how socioeconomic factors may contribute to a complex and overlapping set of social determinants that interact and combine to affect health outcomes. In addition, racial and ethnic biases may amplify associations between SES, social determinants, and health outcomes.

As defined by Healthy People 2020 (Healthy People 2020 website, U.S. Department of Health and Human Services, Office of Disease Prevention and Health Promotion. Healthy People 2020, Social Determinants), “Social determinants of health are conditions in the environments in which people are born, live, work, play, worship, and age that affect a wide range of health, functioning, and quality-of-life outcomes and risks. Conditions (e.g., social, economic, and physical) in these various environments and settings (e.g., school, church, workplace, and neighborhood) have been referred to as “place.” In addition to the more material attributes of “place”, the patterns of social engagement and sense of security and well-being are also affected by where people live. Resources that enhance quality of life can have a significant influence on population health outcomes. Examples of these resources include safe and affordable housing, access to education, public safety, availability of healthy foods, local emergency/health services, and environments free of life-threatening toxins.”

Understanding the complex relationships between social determinants of health and health, functioning, quality-of-life outcomes, cancer outcomes, and cancer risks for the Caribbean population is essential. Examples of social determinants include: availability of resources to meet daily needs (e.g., safe housing and local food markets); access to educational, economic, and job opportunities; access to health care services; and quality of education and job training.

### CONCLUSION

All patents and publications mentioned in this specification are indicative of the levels of those skilled in the art to which the invention pertains. All patents and publications are herein incorporated by reference to the same extent as if each individual publication was specifically and individually indicated to be incorporated by reference. It is to be understood that while a certain form of the invention is illustrated, it is not intended to be limited to the specific form or arrangement herein described and shown. It will be apparent to those skilled in the art that various changes may be made without departing from the scope of the invention and the invention is not to be considered limited to what is shown and described in the specification. One skilled in the art will readily appreciate that the present invention is well adapted to carry out the objectives and obtain the ends and advantages mentioned, as well as those inherent therein.

The systems and methods for electronic coordination of healthcare described herein are presently representative of the preferred embodiments, are intended to be exemplary and are not intended as limitations on the scope. Changes therein and other uses will occur to those skilled in the art which are encompassed within the spirit of the invention. Although the invention has been described in connection with specific, preferred embodiments, it should be understood that the invention as ultimately claimed should not be unduly limited to such specific embodiments. Indeed various modifications of the described modes for carrying out the invention which are obvious to those skilled in the art are intended to be within the scope of the invention.

What is claimed is:

1. A system for electronic coordination of healthcare comprising a digital platform accessible for patients and healthcare providers through the internet, the digital platform including a plurality of tools for navigating a continuum of healthcare services,
wherein the system includes a registry builder to define a registry template into which patient data can be inputted and/or imported to create a data registry.

2. The system according to claim 1, wherein healthcare providers includes any person having an effect on healthcare needs of a patient.

3. The system according to claim 1, wherein the digital platform is supported by architecture comprising a plurality of tiers.

4. The system according to claim 3, wherein the plurality of tiers comprises a web tier, a business tier, a data tier, and an analytics tier.

5. The system according to claim 4, further comprising architecture for security management and for application management.

6. The system according to claim 4, wherein the data tier includes access to external data sources.

7. The system according to claim 6, wherein the external data sources include electronic medical records (EMR), insurance provider databases, and healthcare payer systems.

8. The system according to claim 1, wherein patients using the system have a chronic disease.

9. The system according to claim 8, wherein the chronic disease is cancer or diabetes.

10. The system according to claim 1, wherein the data registry is stored in a data lake and is maintained private until the data registry is shared in the data lake.

11. The system according to claim 10, wherein the data registry is validated by a verification process.

12. A disease registry database created for a chronic disease using the system according to claim 1.

13. The disease registry database according to claim 12, wherein the chronic disease is cancer or diabetes.

14. The disease registry database according to claim 12, wherein the chronic disease is limited to a chronic disease in patients of a specific population.

15. The disease registry database according to claim 12, wherein the data registry is stored in a data lake and is maintained private until the data registry is shared in the data lake.

16. The disease registry database according to claim 15, wherein the data registry is validated by a verification process.
